(19) United States
(12) Patent Application Publication
(10) Pub. No.: US 2025/0108042 A1
(43) Pub. Date: Apr. 3, 2025

(54) Donepezil delivery formulation

(71) Applicant: Davies Pharmaceuticals LLC, Crown Point, IN (US)

(72) Inventor: Robb Davies, Crown Point, IN (US)

(21) Appl. No.: 18/479,245

(22) Filed: Oct. 2, 2023

(51) Int. Cl. A61K 31/445; A61K 9/00; A61K 9/06

(52) U.S. Cl. CPC A61K 31/445; A61K 9/0014; A61K 9/06

(57) ABSTRACT

The treatment of diseases using the solvent described herein to treat many other diseases using dispersion bonding to link the solvent to the medication, which allows easy diffusion of the medication past the layers of the dermis, as a method to deliver each medication to the body and treat each unique disease. The bonds that are created inside the body with each medication are much stronger as dispersion bonding is the weakest form of chemical bonding.

### CROSS-REFERENCE TO RELATED APPLICATIONS

This application claims the benefit of provisional application No. 63/379,049.

### FIELD OF INVENTION

The present disclosure relates to the treatment of Alzheimer's Disease. More specifically, the present disclosure relates to the treatment of Alzheimer's Disease using transdermal therapies and formulations. The invention also relates to the treatment of diseases using the solvent described herein to treat many other diseases using dispersion bonding to link the solvent to the medication, which allows easy diffusion of the medication past the layers of the dermis, as a method to deliver each medication to the body and treat each unique disease. The bonds that are created inside the body with each medication are much stronger as dispersion bonding is the weakest form of chemical bonding.

### BACKGROUND OF THE INVENTION

In 1996, Donepezil was approved for the treatment of dementia in a wide range of Alzheimer's patients, ranging from mild to severe cases. Donepezil has been prescribed and administered to Alzheimer's patients orally, which presents a number of problems. For example, adverse side effects resulting from oral doses of Donepezil commonly include gastrointestinal issues, such as nausea, diarrhea, and vomiting.

Further, oral medications can be particularly problematic for patients suffering from Alzheimer's disease. Due to the loss of cognition and or behavioral changes that afflict many Alzheimer's patients, patients commonly refuse oral medication or spit out medications after they are administered. In an effort to alleviate these issues, transdermal therapies have been investigated. Most recently, a seven-day Donepezil transdermal patch was approved by the FDA for the treatment of Alzheimer's Disease.

However, Donepezil transdermal patches have their own host of problems associated with treatment. Specifically, transdermal patches have been shown to induce skin irritation in the area of application. These application-site reactions can cause itching, redness, and/or irritation. Additionally, as transdermal patches require constant adhesion to the patient's skin for continual dosing, application-site reactions can cause the patient to itch and scratch at the patch. If the patch is removed by the patient because it itches, then the patient is no longer receiving beneficial treatment.

### SUMMARY

In light of this background, there remains a need for improved transdermal therapies utilizing Donepezil as the active ingredient as well as improved methods for the preparation of such therapies.

In the following description, a topical gel is intended to refer to a water-based substance with the consistency commonly referred to as “gel” for cosmetics and topical medications, a topical cream is intended to refer to a substance with consistency and ingredients commonly referred to as “cream” for cosmetics and topical medications, and a “gel-cream” is intended to refer to a substance with properties and ingredients which fall between the two. The following description is also intended to encompass lotions, sprays, and so forth; while the exemplary embodiments are gel, cream, and gel-cream, this disclosure is not intended to be limited to those specific examples. Furthermore, an “aliquot” refers to a single dose of the topical medication described, and “solvent” and “solvent phase” are used to refer to the other substances included in the topical medication, which are chemically compatible with the active ingredient to act as a carrier producing a mixture of sufficiently uniform properties to enable consistent dosing of the patient with the active ingredient.

In certain aspects, the present disclosure relates to improved transdermal therapies and treatments for Alzheimer's disease utilizing Donepezil and improved methods for the preparation of such transdermal therapies and improved methods of delivery of Donepezil. In certain preferred embodiments, the transdermal therapies may be topical gels, topical creams, and or topical gel-creams.

In further aspects, the transdermal therapies include a solvent. The solvent may include compounds such as humectants, emulsifiers, UV light blockers, skin fortifying agents, moisturizing agents, fragrances, antioxidants, water, skin protecting agents, and or nourishing agents, among others. The solvent may further include colorants. Such colorants may be used to specify the dosage of Donepezil of the particular cream and/or gel.

In other aspects, the improved methods of preparing the improved transdermal therapies of the present disclosure may be formulated by preparing the solvent via heating, mixing, and/or filtering. Portions of the solvent may be prepared in separate batches and subsequently mixed together to ensure proper combinations of the ingredients. During solvent preparation, a dosage of Donepezil may be added before the cream and/or gel is cooled and emulsified. Different amounts of Donepezil may be utilized depending on the therapeutic strength of the cream and/or gel being made. The pH of the gel and or cream may be adjusted to ensure there is no skin irritation during application of the therapy.

In certain aspects, an improved method of delivering Donepezil to an Alzheimer's patient is disclosed. In one embodiment, the transdermal cream/gel is applied to the skin, which overlays skeletal muscle, of an Alzheimer's patient. To receive a proper daily dose of the Donepezil, the patient, caretaker, and/or healthcare provider may utilize a stronger dose, also said as a higher concentration, cream/gel once daily. In other embodiments, the patient may apply a lower dose or concentration cream/gel two or more times throughout the day. The Donepezil is adsorbed through the skin, which provides the needed treatment to improve cognition and/or behavior to the patient. Meanwhile, the solvent phase (or non Donepezil ingredients, also called inactive ingredients or carrier ingredients) of the cream/gel will help to moisturize, nourish, fortify, protect, and heal the skin to reduce skin irritation at the application site.

Additional embodiments of the invention, as well as features and advantages thereof, will be apparent from the descriptions herein.

### BRIEF DESCRIPTION OF DRAWINGS

FIG. 1 depicts a cross-sectional view of human skin with an embodiment of the present disclosure applied thereto.

### DETAILED DESCRIPTION OF THE INVENTION

For the purposes of promoting and understanding the principles of the invention, reference will now be made to the embodiment illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

Gels, creams, and gel-creams of the present disclosure include a solvent phase or carrier used to deliver the active ingredient, Donepezil. The solvent, also called solvent phase or carrier, may be comprised of a number of different types of compounds. For example, the solvent may include humectants, moisturizing agents, UV light blockers, skin and muscle fortifying agents, nourishing agents, emulsifiers, sources of antioxidants, fragrances, and/or water, among others. The solvent phase is formulated to properly deliver Donepezil, while reducing adverse side effects of other delivery methods, such as irritated skin at the application site.

The solvent of the transdermal therapy of the present disclosure may include one or more humectants. Humectants absorb and maintain moisture from the surrounding air. Humectants come in a variety of types. For example, humectants may be natural, naturally derived, or synthetically made. Specifically, humectants which may be used include beeswax, aloe vera, hyaluronic acid, petroleum gel, honey, and glycols, among others. The solvent phase may include one or more of these humectants and combinations thereof.

The solvent may include skin and muscle fortifying/anti-inflammatory agents. Beeswax and Vitamin E are also strong fortifying/anti-inflammatory agents for skin and muscles.

In addition, the solvent of the transdermal therapies of the present invention may include moisturizing agents included to assist in the repair of damaged or dry skin. For example, the solvent may include glycerine. The glycerine has the added benefit of maintaining the aroma of any fragrance added to the solvent and acting as an emulsifying agent. Alternatively or additionally, the solvent may include petroleum gel. The solvent may also include antioxidants and/or a source of antioxidants. For example, the solvent may include Vitamin A, Vitamin C, Vitamin E, and/or other antioxidants. In other embodiments, the solvent may include sources of antioxidants, such as avocado oil.

In addition, the formulation may have skin repairing, nourishing, and protecting agents. Roses, beeswax, and aloe vera also help to repair irritated or damaged skin. Vitamin E is used to nourish and protect skin cells from damage.

There may also be ingredients to protect skin specifically against UV light. For example, Beeswax may provide mild to intense UV light protection.

The solvent may further include a source of fragrance. In some embodiments, the fragrance may be a pre-formulated product/synthetic fragrance added during preparation. In other embodiments, the fragrance may be a naturally occurring or naturally derived fragrance. For example, essential oils, extracts, or components of flowers, such as rose petals, may be used to impart a pleasant fragrance.

The solvent may also include a colorant. The colorants may be used to designate the therapeutic strength of the transdermal therapy of the present disclosure. For example, a colorless/white cream or gel may indicate that application of a particular size (e.g. 2-3 ml) aliquot will deliver a 5 mg dose of Donepezil, while a light blue cream may indicate delivery of a 10 mg dose of Donepezil, while a light blue cream or gel may indicate delivery of a 10 mg dose of Donepezil when using the same size aliquot (2-3 ml). Such color-coded creams/gels will help ensure that patients, caregivers, and/or healthcare professionals are applying the proper amount of cream/gel that is required for the patient to receive a particular dosage.

Other ingredients may also be utilized in the creams and gels of the present disclosure. For example, water and or rose-water may be used.

As will be understood by one of ordinary skill in the art to which the present disclosure relates, various quantities, ratios, and or techniques may be utilized in the practice of the present disclosure.

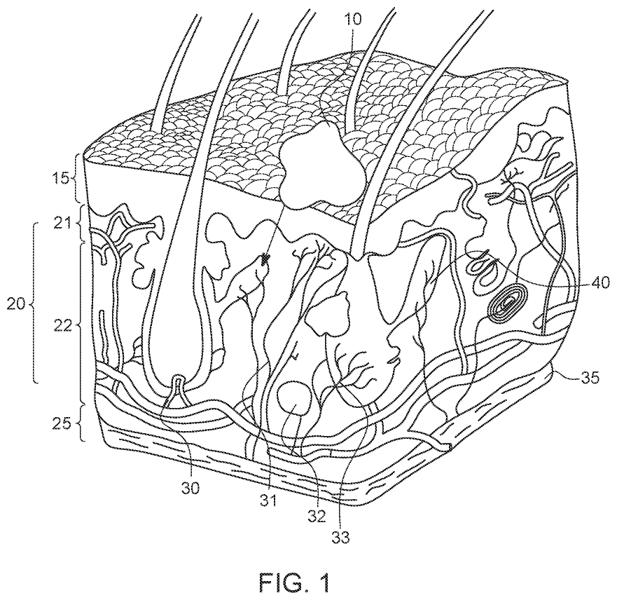

FIG. 1 depicts a cross-sectional view of human skin with an aliquot 10 of an embodiment of the present disclosure applied thereto. The human skin consists of three layers: the epidermis 15, the dermis 20, and the hypodermis 25. The dermis 20 can be further subdivided into the papillary layer 21 and the reticular layer 22. Within the layers of the human skin are nervous system structures 30 and muscles, for example, the arrector pili muscle 40. The nervous system structure 30 includes sensory nerve fibers 31, Pacinian corpuscle 32, and hair follicle receptors 33. Below the three layers of human skin lies the skeletal muscle 35.

As shown in FIG. 1, aliquot 10 of an embodiment of the present disclosure is applied to the epidermis 15. The aliquot 10 is absorbed into the skin, providing any moisturizing, nourishing, protective, and or fragrance properties contained in the aliquot 10. As such, the application site will not become irritated, etc. Further, the Donepezil (not shown separately) is delivered to the skeletal muscular junction where it prevents acetylcholinesterase from breaking down acetylcholine. The additional acetylcholine present then allows for better communication between nerve cells. The excess breakdown of acetylcholine is thought to cause the dementia and or behavioral symptoms associated with Alzheimer's disease.

In order to promote a further understanding of the present invention and its various embodiment, the following specific examples are provided. It will be understood that these examples are illustrative and not limiting of the invention.

### Example 1

### Preparation of Donepezil Transdermal Therapy Rose Gel-Cream

### Materials and Methods

The preparation of this exemplar cream utilizes two vessels or tanks. The two vessels or tanks may be prepared simultaneously or consecutively. However, the contents of Tank 1 and Tank 2 will be added together. For the preparation of Tank 1, water and rose petals are added to a clean tank. The rose petals and water are mixed and the contents are heated for thirty minutes at 50 degrees C. Once the rose petals are wilted, the tank is then heated to approximately 90 plus or minus 5 C, with continuous mixing. This creates rose-water.

For the preparation of Tank 2, avocado oil is added to the Tank and mixed. The tank is then heated to 70 degrees C. Once the Tank reaches temperature, beeswax is added with continuous mixing. The mixing will continue until the contents of Tank 2 are homogeneous.

Once Tank 1 and Tank 2 are properly prepared, the contents of each are mixed together. This is accomplished by filtering the contents of Tank 1 through an 80-mesh sieve and pumping the filtered Tank 1 material into Tank 2. Once the contents of Tank 1 are transferred to Tank 2. Glycerine is then added to Tank 2 with continued mixing and heating to 70 degrees C., to be used as an emulsifier to homogenize the mixture of components within Tank 2. After fifteen minutes, the correct dosage of Donepezil is added to the mixture. The contents in Tank 2 are mixed for an additional fifteen minutes to ensure homogeneity. After completion of this mixing step, the contents are continuously mixed and cooled to room temperature until the solution achieves a semi-sold state. Colorants and/or fragrance may be added to the gel-cream with additional mixing.

If necessary, the pH of the resulting gel-cream can be adjusted. Ideally, the pH of the gel-cream is around 4.5 to 4.4, the approximate pH of human skin. Adjustment of the pH of the solution can be accomplished using either Sodium Hydroxide (Na2OH) or Citric Acid, as needed. Citric acid or Sodium Hydroxide may also be added after the addition of Donepezil to maintain consistency of the product.

The amounts of each compound or ingredient in the compound will vary depending on the desired dosage of Donepezil that is to be included in the resulting gel=cream. Additionally, the heating and mixing may be accomplished by any suitable means known to the hose skilled in the art in this field (e.g., a heating plate/mixing plate for small batches or industrial sized heaters and mixers for large batches).

### Example 2

### Preparation of Donepezil Transdermal Therapy Gel

### Materials and Methods

Petroleum gel is added to a mixing tank and heated to 100 C to ensure the petroleum gel liquefies. After 30 minutes of mixing, Glycerine is added to the mixing tank. The tank is mixed for an additional 30 minutes, then the appropriate amount of Donepezil is added to achieve the target concentration of the batch. The tank is then cooled to 35 C and Vitamin E extract is added. The solution is then mixed for at least 30 minutes. If desired, fragrance and/or colorants can be added to the solution at this point. Then, the solution is continuously mixed and cooled to room temperature until a gel consistency is achieved.

If necessary, the pH of the resulting gel can be adjusted. Ideally, the pH of the gel is around 4.5 to 5.5, the approximate pH of human skin. Adjustment of the pH of the solution can be accomplished using either Sodium Hydroxide (NaOH) or Citric Acid, as needed. Citric acid or Sodium Hydroxide may also be added after the addition of Donepezil to maintain consistency of the product.

The amounts of each compound or ingredient utilized in the preparation of Example 2 will vary depending on the strength/dosage of Donepezil that is desired for the batch.

### Example 3

### Method of Delivery Utilizing Example 1 or Example 2

### Materials and Methods

The transdermal therapy compounds from Example 1 or Example 2 are used for the treatment of Alzheimer's Disease. A patient presents with both cognitive deterioration and behavioral issues as a result of Alzheimer's Disease. A 2-3 mL aliquot of the transdermal therapy is applied to the patient's skin, preferably on their arm, thigh, buttocks, or back, which overlays skeletal muscle, daily using a formulation of the required strength (i.e., 10 mg formulation for a 10 mg prescription). Within 2-3 days after daily application of the transdermal therapy, cognitive and behavioral symptoms are lessened. Within 3-5 weeks of daily application of the transdermal therapy, patient's symptoms are lessened further. Example 1 will add UV light protection. Examples 1 and 2 will also reduce/eliminate any skin irritation or itching, burns, joint pain, or wounds by the inclusion of the moisturizing agents, nourishing agents, UV light protection agents, fortifying agents, skin protecting agents, humectants, and or antioxidants present in the transdermal therapy.

### Example 4

### Method of Delivery of Donepezil Utilizing Example 1 or Example 2

### Materials and Methods

The transdermal therapy compounds from Example 1 or example 2 are used for the treatment of Alzheimer's Disease. A patient presents with both cognitive deterioration and behavioral issues as a result of Alzheimer's disease. A 2-3 mL aliquot of the transdermal therapy is applied to the patient's skin, preferably their arm, thigh, buttocks, or back, which overlays skeletal muscle, twice daily using a formulation of half of the required strength (i.e. 5 mg formulation for a 10 mg prescription). The transdermal therapy is applied both in the morning and at night. Within 2-3 days after twice daily application of the transdermal therapy, cognitive and behavioral symptoms are lessened. Within 3-5 weeks of twice daily application of the transdermal therapy, patient's symptoms are lessened further. Example 1 will add UV light protection. Example 1 and 2 will also reduce/eliminate any skin irritation, itching, or joint pain by the inclusion of the moisturizing agents, nourishing agents, fortifying agents, skin protecting agents, humectants, and or antioxidants present in the transdermal therapy.

Note that the aliquot size used to administer an appropriate dose is dependent upon the concentration (i.e. mg/mL) of Donepezil in the transdermal therapy and how it is prepared. The aliquot sizes in Examples 3 and 4 are illustrative.

While the invention has been illustrated and described in detail in the foregoing description, the same is to be considered as illustrative and not restrictive in character, it being understood that only the preferred embodiment has been shown and described and that all changes and modifications that come within the spirit of the invention are desired to be protected. In addition, all references cited herein are indicative of the level of skill in the art and are hereby incorporated by reference in their entirety.

1. A transdermal therapy for patients comprising donepezil and a solvent.

2. The transdermal therapy for patients of claim 1, wherein the transdermal therapy is a gel, a cream, or a gel-cream.

3. The transdermal therapy for patients of claim 1, wherein the solvent comprises at least one of a humectant, an emulsifier, a UV light blocker, a skin fortifying agent, a moisturizing agent, a fragrance, an antioxidant, water, a skin protecting agent, and a nourishing agent.

4. The transdermal therapy for patients of claim 1, wherein the solvent further comprises a coloring for use in conveying information regarding the concentration of medication in the transdermal therapy.

5. A method of applying a transdermal therapy for patients, comprising administering the transdermal therapy of claim 1 once a day to a section of skin above skeletal muscle.

6. A method of applying a transdermal therapy for patients, comprising administering the transdermal therapy of claim 1 twice a day to a section of skin above skeletal muscle.

7. The transdermal therapy of claim 1, wherein the composition of the solvent is such that the solvent can be used as a delivery method for other medications by linking to the other medications to the solvent via dispersion bonding.
